SEQUENCE LISTING (2 sequences)

SEQ ID NO: 1 - protein, 449 aa, synthetic construct
  source 1..449: note = a heavy chain of obinutuzumab
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, synthetic construct
  source 1..219: note = a light chain of obinutuzumab
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0026847 A1
(43) Pub. Date: Jan. 23, 2025

(54) TREATMENT OF CD20-POSITIVE B-CELL LYMPHOMA WITH OBINUTUZUMAB

(71) Applicant: F. Hoffmann-La Roche AG, Basel (CH)

(72) Inventors: Michael WENGER, Basel (CH); Mehrdad MOBASHER, South San Francisco, CA (US); Chin-Yu LIN, South San Francisco, CA (US)

(21) Appl. No.: 18/893,101

(22) Filed: Sep. 23, 2024

Related U.S. Application Data
(63) Continuation of application No. 16/756,057, filed on Apr. 14, 2020, now Pat. No. 12,129,304.
(60) Provisional application No. 62/574,297, filed on Oct. 19, 2017.

(51) Int. Cl. C07K 16/28; A61K 39/00; A61K 45/06; A61K 47/10; A61K 47/18; A61P 35/00

(52) U.S. Cl. CPC C07K 16/2887; A61K 47/10; A61K 47/183; A61P 35/00; A61K 2039/505; A61K 2039/545; A61K 45/06

(57) ABSTRACT

The present invention relates to administration speed of obinutuzumab.

### SEQUENCE LISTING

The instant application contains a Sequence Listing which has been submitted electronically in XML format and is hereby incorporated by reference in its entirety. Said XML copy, created on Aug. 5, 2024, is named “51485-002002_Sequence_Listing_8_5_24” and is 3,642 bytes in size.

### TECHNICAL FIELD

The present invention relates to administration speed of obinutuzumab.

### BACKGROUND ART

Obinutuzumab is a glycoengineered, type II anti-CD20 monoclonal antibody indicated for the treatment of B-cell malignancies. It differs from its predecessor rituximab by having lower complement-dependent cytotoxicity but enhanced antibody-dependent cytotoxicity and direct B-cell death (NPL1-3, FIG. 1). In the phase III GALLIUM trial, which compared chemotherapy combined with either obinutuzumab or rituximab followed by anti-CD20 antibody maintenance therapy, obinutuzumab-based immunochemotherapy resulted in a clinically meaningful improvement in pro-gression-free survival in patients with previously untreated follicular lymphoma (FL) (NPL4, 5). Obinutuzumab plus bendamustine followed by obinutuzumab maintenance also improved efficacy over bendamustine monotherapy in rituximab-refractory patients with indolent B-cell non-Hodgkin lymphoma (NHL) in the phase III GADOLIN study (NPL 6).

### CITATION LIST

### Non Patent Literature

[NPL1] Mossner E, Brunker P, Moser S, et al. Increasing the efficacy of CD20 antibody therapy through the engineering of a new type II anti-CD20 antibody with enhanced direct and immune effector cell-mediated B-cell cytotoxicity. Blood 2010; 115:4393-402.
[NPL2] Herter S, Herting F, Mundigl O, et al. Preclinical activity of the type II CD20 antibody GA101 (obinutuzumab) compared with rituximab and ofatumumab in vitro and in xenograft models. Mol Cancer Ther 2013; 12:2031-42.
[NPL3] Tobinai K, Klein C, Oya N, Fingerle-Rowson G. A review of obinutuzumab (GA101), a novel type II anti-CD20 monoclonal antibody, for the treatment of patients with B-cell malignancies. Adv Ther 2017; 34:324-56.
[NPL4] Marcus R, Davies A, Ando K, et al. Obinutuzumab for the first-line treatment of follicular lymphoma. N Engl J Med 2017; 377:1331-44.
[NPL5] Hiddemann W, Barbui A M, Canales Albendea M A, et al. Immunochemotherapy with obinutuzumab or rituximab in previously untreated folllicular lymphoma in the randomised phase III GALLIUM study: analysis by chemotherapy regimen. Hematol Oncol 2017; 35:117-9.
[NPL6] Sehn L H, Chua N, Mayer J, et al. Obinutuzumab plus bendamustine versus bendamustine monotherapy in patients with rituximab-refractory indolent non-Hodgkin lymphoma (GADOLIN): a randomised, controlled, open-label, multicentre, phase 3 trial. Lancet Oncol 2016; 17:1081-93.

### SUMMARY OF INVENTION

Obinutuzumab is currently given by intravenous (abbreviated as ‘IV’ in this description) infusion. Lengthy and/or frequent IV infusions are burdensome and inconvenient for patients and result in the need for lengthy observation times with increased nursing and administration staff workloads. Regular IV infusion (abbreviated as ‘RI’ in this description) of obinutuzumab takes approximately 3 to 4 h, and it is reasonable to consider that reducing the duration of infusion has potential advantages in terms of patient convenience, and more efficient use of healthcare facilities and staff time (FIG. 2). The main potential disadvantage of a shorter duration of infusion (abbreviated as ‘SDI’ in this description) lies in the possibility of increased risk of infusion related reactions (IRRs) mediated by cytokine release. However, studies in patients with rheumatoid arthritis or B-cell NHL have shown reduction of rituximab infusion times from at least 4 h to 1.5-2 h to be feasible, which has in turn led to the recommendation to increase infusion rates for rituximab and, similarly, to the investigation of SDI in patients receiving obinutuzumab.

SDI was also investigated in the GATHER trial, a phase II, open-label, multicenter, single-arm study of obinutuzumab in combination with cyclophosphamide, doxorubicin, vincristine and prednisolone (abbreviated as ‘CHOP’ in this description, where prednisolone is exchangeable for prednisone) chemotherapy in 80 previously untreated patients with CD20-positive advanced diffuse large B-cell lymphoma (abbreviated as ‘DLBCL’ in this description). Both of the SDI times evaluated in GATHER, 120 and 90 min, were well tolerated, with no IRRs of grade 3. Overall, 4% of the GATHER population was of Asian ethnicity, and ethnic differences in the frequency of polymorphisms in genes involved in drug metabolic pathways have been suggested to be associated with changes in enzyme activity that might affect drug pharmacokinetics (abbreviated as ‘PK’ in this description). However, data obtained in various geographic populations receiving obinutuzumab have shown no relevant differences in the PK of obinutuzumab in Asian (including Chinese and Japanese) and non-Asian patients.

To explore these concepts further, the phase II GATS study (J029737, JapicCTI-152 848) was carried out to investigate the tolerability of obinutuzumab given using SDI in previously untreated patients with CD20-positive B-cell NHL, in particular the rate of grade IRRs, and to evaluate serum obinutuzumab concentrations and PK, and the time course of cytokine release (FIG. 3).

Specifically, the present invention relates to:

[1] A pharmaceutical composition for treating CD20-positive B-cell lymphoma comprising obinutuzumab, which is intravenously drip infused at 1000 mg of obinutuzumab per administration, and given according to the administration speeds of the following (a) and (b) in two or more cycles:
(a) the maximum administration speed in the first cycle is equal to or more than 200 mg of obinutuzumab an hour, preferably equal to or more than 300 mg of obinutuzumab an hour, more preferably equal to or more than 400 mg of obinutuzumab an hour;
(b) the maximum administration speed in the second or later cycle is equal to or more than 700 mg of obinutuzumab an hour, preferably equal to or more than 800 mg of obinutuzumab an hour, more preferably equal to or more than 900 mg of obinutuzumab an hour.

[2] The pharmaceutical composition according to [1], wherein a duration per administration in the second or later cycle is within 180 minutes, preferably within 150 minutes, more preferably within 120 minutes, the most preferably within 90 minutes.

[3] The pharmaceutical composition according to [1] or [2], which is administered 3 times in the first cycle, and once a cycle in the second or later cycle.

[4] The pharmaceutical composition according to [3], wherein the first administration in the first cycle is initiated at a speed of 50 mg of obinutuzumab an hour, and the second or later administration in the first cycle is initiated at a speed of 100 mg of obinutuzumab an hour.

[5] The pharmaceutical composition according to any one of [1] to [4], wherein the administration speed in the second or later cycle is increased to 700 mg of obinutuzumab an hour or faster, preferably to 800 mg of obinutuzumab an hour or faster, more preferably up to 900 mg of obinutuzumab an hour.

[6] The pharmaceutical composition according to any one of claims 1 to 5, wherein, in (b), the pharmaceutical composition is given according to at least one of the following (c) to (e) conditions:
(c) if no infusion reaction of Grade 3 or above appeared with the last three administrations, and the number of lymphocytes in peripheral blood before administration is less than 5000/μL, administration is carried out at 100 mg/hour for 30 minutes. If no infusion reaction is observed all that time, the speed can be increased to 900 mg/hour. Depending on the condition of the patient, the speed is decreased to, for example, the administration speed in cycle 1, as appropriate.
(d) if an infusion reaction of Grade 1/2 appeared, administration is restarted at half the speed before administration was stopped. If no infusion reaction is observed in 30 minutes, the speed can be increased to 900 mg/hour.
(e) if an infusion reaction of Grade 3, administration is restarted at 200 mg/hour or lower. If no infusion reaction is observed in 30 minutes, the speed can be increased by 50 mg/hour every 30 minutes to a maximum of 400 mg/hour.

[7] The pharmaceutical composition according to any one of [1] to [6], which is administered on days 1, 8 and 15 in the first cycle, and on day 1 in the second or later cycle.

[8] The pharmaceutical composition according to any one of [1] to [7], wherein each cycle is 3 weeks.

[9] The pharmaceutical composition according to any one of [1] to [7], which is used in combination with at least one of other anti-tumor agents, and whose administration cycle is synchronized with a dosing cycle of said at least one of other anti-tumor agents, wherein the dosing cycle is 4 weeks a cycle.

[10] The pharmaceutical composition according to [9], wherein said at least one of other anti-tumor agents is selected from CHOP, CVP, bendamustine, fludarabine, lenalidomide, an anti-PD-1 antibody, and an anti-PD-L1 antibody.

[11] The pharmaceutical composition according to any one of [1] to [10], wherein the pharmaceutical composition is given every two months for two years as maintenance monotherapy after said two or more cycles.

[12] The pharmaceutical composition according to any one of [1] to [11], wherein the obinutuzumab concentration in infusion fluid when intravenously drip infused is 10 to 40 mg/mL, preferably 20 to 30 mg/mL, more preferably 25 mg/mL.

[13] The pharmaceutical composition according to any one of [1] to [12], further comprising a trehalose hydrate, L-histidine, L-histidine hydrochloride hydrate, or polyoxyethylene (160) polyoxypropylene (30) glycol as an additive.

[14] Use of obinutuzumab in a manufacture of a pharmaceutical composition for treating CD20-positive B-cell lymphoma comprising obinutuzumab, wherein the composition is intravenously drip infused at 1000 mg of obinutuzumab per administration, and given according to the administration speeds of the following (a) and (b) in two or more cycles:
(a) the maximum administration speed in the first cycle is equal to or more than 200 mg of obinutuzumab an hour, preferably equal to or more than 300 mg of obinutuzumab an hour, more preferably equal to or more than 400 mg of obinutuzumab an hour;
(b) the maximum administration speed in the second or later cycle is equal to or more than 700 mg of obinutuzumab an hour, preferably equal to or more than 800 mg of obinutuzumab an hour, more preferably equal to or more than 900 mg of obinutuzumab an hour.

[15] A method for treating CD20-positive B-cell lymphoma by a pharmaceutical composition comprising obinutuzumab, wherein the composition is intravenously drip infused at 1000 mg of obinutuzumab per administration, and given according to the administration speeds of the following (a) and (b) in two or more cycles:
(a) the maximum administration speed in the first cycle is equal to or more than 200 mg of obinutuzumab an hour, preferably equal to or more than 300 mg of obinutuzumab an hour, more preferably equal to or more than 400 mg of obinutuzumab an hour;
(b) the maximum administration speed in the second or later cycle is equal to or more than 700 mg of obinutuzumab an hour, preferably equal to or more than 800 mg of obinutuzumab an hour, more preferably equal to or more than 900 mg of obinutuzumab an hour.

### BRIEF DESCRIPTION OF DRAWINGS

FIG. 1 shows characters of obinutuzumab. Obinutuzumab is a glycoengineered type II anti-CD20 mAb and has greater direct cell death induction and ADCC/ADCP activity than rituximab. ADCC, antibody-dependent cell-mediated cytotoxicity; ADCP, antibody-dependent cellular phagocytosis.

FIG. 2 shows length of administration time and risk of IRR. Considering a heavy strain on patients, realization of SDI will increase the clinical usefulness of obinutuzumab. In phase 1 study (J021900 study), which evaluated the safety, tolerability, PK and preliminary efficacy of obinutuzumab in Japanese patients, all 12 patients who were administered obinutuzumab experienced infusion-related reaction (IRR) at Cycle 1 Day 1.

FIG. 3 shows summary of GATS study.Objective, to confirm acceptability of obinutuzumab shorter duration of infusion (SDI) in Japanese patients. Target population, previously untreated patients with CD20-positive B-cell NHL (DLBCL, FL, MZL). Study design, Phase II, Multicenter, Open-label, Single-arm, G-CHOP×8 Cycle. Primary endpoint, Incidence rate of ≥Grade 3 infusion-related reactions in Cycle 2; Serum concentrations and pharmacokinetic parameters of obinutuzumab; time course of cytokines (TNFα, IFNγ, IL-6, IL-8, IL-10). Target number of patients, 36 (enrolled).

FIG. 4 shows clinical design of GATS study. Study drug administration and SDI inclusion criteria were designed on the basis of GATHER study, which evaluated the safety and efficacy of G-CHOP, and SDI was confirmed by GATS study. Obinutuzumab was administered by regular infusion with CHOP in day 1 of cycle 1, for 4 hours and 15 minutes, and in day 8 and 15 of the cycle 1, for 3 hours and 15 minutes. If patients meet the SDI inclusion criteria (at least 3 consecutive doses of obinutuzumab by regular infusion without any ≥Grade 3 infusion-related reactions; pre-SDI peripheral lymphocyte count <5000/μL), obinutuzumab was administered by SDI in cycle 2 to 8.

FIG. 5 shows comparison of administration speed between regular infusion and SDI in GATS study.

FIG. 6 shows disposition of patients in GATS study.

FIG. 7 shows patient characteristics in GATS study.

FIG. 8 shows safety profile in GATS study. No ≥Grade 3 infusion-related reactions occurred in the SDI transition patients. The safety profile under SDI was comparable to that under regular infusion.

FIG. 9 shows number of patients with IRR in GATS and GATHER study. The same tendencies were shown between GATS and GATHER study. The same tendencies were shown between GATS and GATHER studies.

FIG. 10 shows obinutuzumab PK in Japanese patients after administration by SDI in GATS study versus regular infusion in GOYA study. The serum obinutuzumab concentrations of the SDI transition patients in GATS study followed a similar time course to that of patients under regular infusion in the GOYA study.

FIG. 11 shows obinutuzumab PK in SDI patients of GATS study and GATHER study. No ethnic difference was observed considering individual variability, even though timing of blood sampling was differed between GATS and GATHER studies. Serum concentration just after cycle 2 under SDI was similar to that in cycle 8, therefore, PK reached a steady state at cycle 2 and was not affected by SDI.

FIG. 12 shows IL-6 release after administration of obinutuzumab in GATS study. The peaks of cytokine increase were in infusion in day 1 of cycle 1 and they quickly decreased by 2-5 hours after the infusion. No marked changes were found after starting SDI. The similar tendencies were observed in TNFα, IFNγ, IL-8, and IL-10.

FIG. 13 shows treatment efficacy of obinutuzumab to DLBCL and FL in GATAS study.

FIG. 14 shows acceptability for SDI of obinutuzumab on the basis of GATS study. No ≥Grade 3 IRR occurred in the SDI transition patients. IRR occurred most commonly on day 1 of cycle 1 under regular infusion, but all were Grade 1 or 2. 3 IRR were observed under SDI in cycle 6, 7, and 8, but all were classified as Grade 1. Similar tendency was observed in GATHER study. Administration time and ethnic differences do not seem to have any effect on the PK of obinutuzumab. Cytokine elevation was observed during the first obinutuzumab infusion, but immediately decreased at the end of the infusion. As a whole, SDI of obinutuzumab was shown to be acceptable.

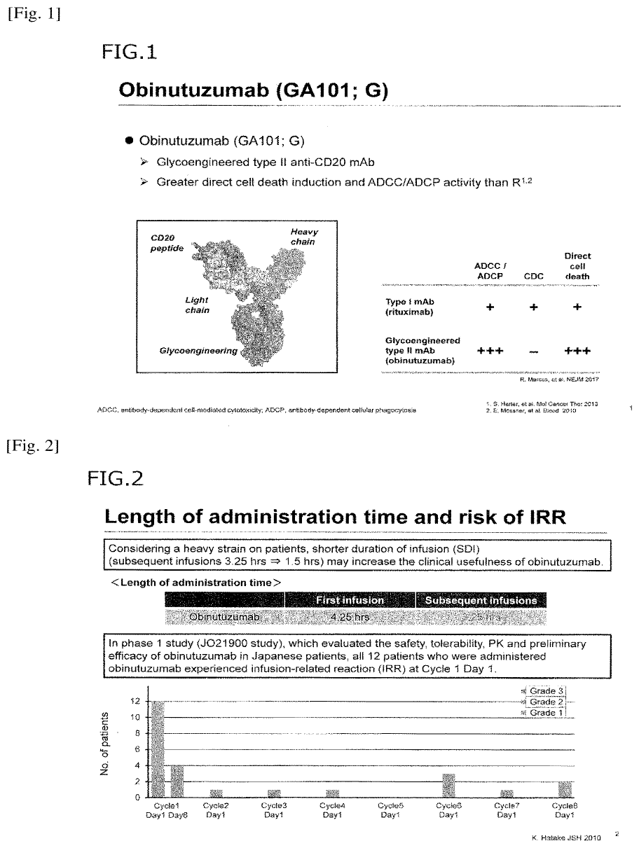

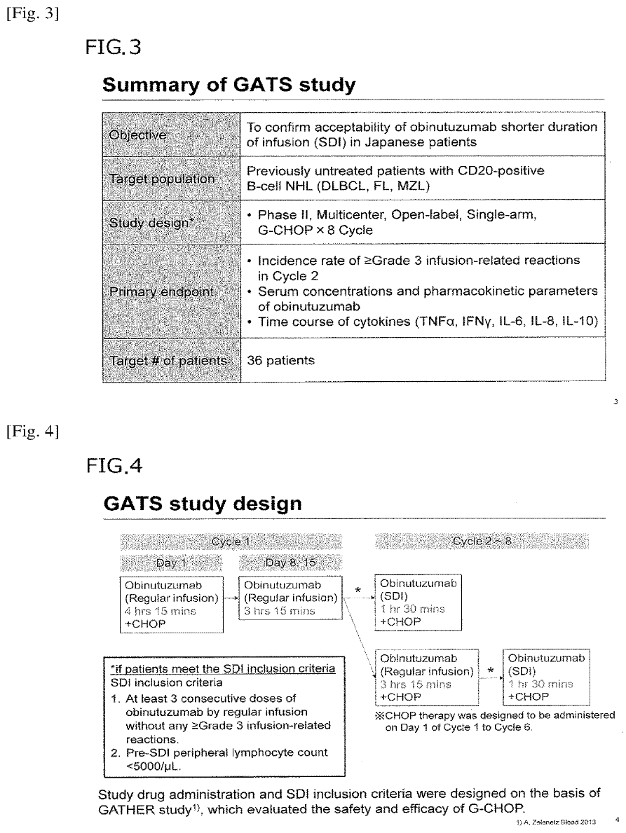

### DETAILED DESCRIPTION OF THE INVENTION

### Description of Embodiments

### I. Summary Regarding Dosage and Administration of Obinutuzumab.

In the present application, obinutuzumab is a glycoengineered, genetically recombined and humanized anti-CD20 monoclonal antibody, a glycoprotein that exhibits the characteristics of a type II anti-CD20 antibody and comprises two heavy chains of 449 amino acid residues and two light chains of 219 amino acid residues, and has a molecular weight of about 148,000-150,000. Specifically, obinutuzumab in the present application includes not only those specified under “Obinutuzumab (Genetical Recombination)” in Japanese Accepted Names for Pharmaceuticals (JAN) but also biosimilar and biobetter products thereof.

Below, a pharmaceutical composition comprising obinutuzumab shall be referred to as “present formulation”.

### <Effects and Efficacy>

Examples of the “effects and efficacy” of the present formulation are provided below as one embodiment.
An example of a disease targeted by the present formulation is CD20-positive B-cell lymphoma. That is, the present formulation is a pharmaceutical composition for treating CD20-positive B-cell lymphoma comprising obinutuzumab. Examples of CD20-positive B-cell lymphoma include follicular lymphoma, low-grade lymphoma, intermediate-grade lymphoma, and high-grade lymphoma. The CD20-positive B-cell lymphoma is preferably CD20-positive B-cell follicular lymphoma.

### <Dosage and Administration>

Examples of the “dosage and administration” of the present formulation are provided below as one embodiment. The dosage and administration tolerability, including administration speed, illustrated below, has been demonstrated by the GATS study.
Normally, obinutuzumab is intravenously drip infused at 1000 mg per administration in adults.
For induction therapy, each cycle is three weeks, and administration is performed on days 1, 8 and 15 in cycle 1, and on day 1 in cycles 2 to 8. Where an anti-tumor agent is used in combination and is administered in four-week intervals, each cycle is four weeks, and administration is performed on days 1, 8 and 15 in cycle 1, and on day 1 in cycles 2 to 6.
After 24 weeks of induction therapy, maintenance therapy is carried out by administering the present formulation alone every two months for two years.
The present formulation is administered at the following administration speed. Moreover, grading criteria are in accordance with NCI-CTCAE ver. 4.03.

### (On First Administration)

Intravenous drip infusion is initiated at a speed of 50 mg/hour. The speed is increased by 50 mg/hour every 30 minutes while the patient's condition is adequately monitored, and can be raised to a maximum of 400 mg/hour.

(On or after Second Administration in Cycle 1)

If no infusion reaction of Grade 2 or above appeared with the previous administration, administration is initiated at 100 mg/hour, and if no infusion reaction is observed, the speed can be increased by 100 mg/hour every 30 minutes to a maximum of 400 mg/hour.

(In or after Cycle 2)

If no infusion reaction of Grade 3 or above appeared with the last three administrations, and the number of lymphocytes in peripheral blood before administration is less than 5000/μL, administration is carried out at 100 mg/hour for 30 minutes. If no infusion reaction is observed, the speed can be increased to 900 mg/hour. Depending on the condition of the patient, the speed is decreased to, for example, the administration speed in cycle 1, as appropriate.
If an infusion reaction appeared, the following response is taken.

### (If an Infusion Reaction of Grade 2 or Below Appeared)

Administration is stopped or the administration speed is decreased.

### (If an Infusion Reaction of Grade 3 Appeared)

Administration is stopped and appropriate measures are taken.

If administration was stopped, after the patient has recovered/remitted from the infusion reaction, the administration speed is adjusted and administration is restarted as described below.

### (If an Infusion Reaction of Grade 4 Appeared)

Administration of the present formulation is stopped immediately, and appropriate measures are taken.

### (If an Infusion Reaction of Grade 3 Recurred and if an Infusion Reaction of Grade 4 Appeared)

The present formulation will not be administered again.
When administration is restarted after administration was stopped following an infusion reaction, the administration speed at the time of restart is handled as follows.
   (On First Administration and on or after Second Administration in Cycle 1)

Administration is restarted at half the speed before administration was stopped. If no infusion reaction is observed in 30 minutes, the speed can be increased by 50 mg/hour every 30 minutes to a maximum of 400 mg/hour.

(If an Infusion Reaction of Grade 1/2 Appeared in or after Cycle 2)

Administration is restarted at half the speed before administration was stopped. If no infusion reaction is observed in 30 minutes, the speed can be increased to 900 mg/hour.

(If an Infusion Reaction of Grade 3 Appeared in or after Cycle 2)

Administration is restarted at 200 mg/hour or lower. If no infusion reaction is observed in 30 minutes, the speed can be increased by 50 mg/hour every 30 minutes to a maximum of 400 mg/hour.

### II. Pharmaceutical Composition

A pharmaceutical composition in the present invention comprises obinutuzumab. In one embodiment, the pharmaceutical composition comprises a pharmaceutically effective amount of obinutuzumab. Obinutuzumab is a glycoengineered, genetically recombined and humanized anti-CD20 monoclonal antibody, a glycoprotein that exhibits the characteristics of a type II anti-CD20 antibody and comprises two heavy chains of 449 amino acid residues and two light chains of 219 amino acid residues, and has a molecular weight of about 148,000-150,000. Specifically, obinutuzumab in the present application includes not only those specified under “Obinutuzumab (Genetical Recombination)” in Japanese Accepted Names for Pharmaceuticals (JAN), but also biosimilar in which the amino acid sequence of a heavy chain is revealed in SEQ No. 1 and the amino acid sequence of a light chain is revealed in SEQ No. 2, and biobetter products thereof originating from those amino acids sequences.

The pharmaceutical composition is used for treating CD20-positive B-cell lymphoma comprising. Examples of the CD20-positive B-cell lymphoma include follicular lymphoma, low-grade lymphoma, intermediate-grade lymphoma, and high-grade lymphoma. The CD20-positive B-cell lymphoma is preferably CD20-positive B-cell follicular lymphoma.

In one embodiment, the concentration of obinutuzumab in the pharmaceutical composition as infusion fluid when intravenously drip infused is normally 10 to 40 mg/mL. In another embodiment, the concentration is 20 to 30 mg/mL. In another embodiment, the concentration is 25 mg/mL. In a preferred embodiment, the concentration is 20 to 30 mg/mL. In a more preferred embodiment, the concentration is 25 mg/mL.

In one embodiment, the pharmaceutical composition may further comprise at least one additive selected from a trehalose hydrate, L-histidine, L-histidine hydrochloride hydrate, or polyoxyethylene (160) polyoxypropylene (30) glycol. In a preferred embodiment, the pharmaceutical composition comprise a trehalose hydrate, L-histidine, L-histidine hydrochloride hydrate, and polyoxyethylene (160) polyoxypropylene (30) glycol as additives.

In one embodiment, the pharmaceutical composition is administered in two or more cycles. A period of the cycle may be decided in 3 to 5 weeks by one of ordinary skill in the art. Example of the period is 3 weeks or 4 weeks. In the case that the pharmaceutical composition is administered as monotherapy, the period is preferably 3 weeks. In the case that the pharmaceutical composition is administered in combination with at least one of anti-tumor agents, the period is preferably 4 weeks. When the pharmaceutical composition is combined with such anti-tumor agent(s), administration cycles of the pharmaceutical composition are preferably synchronized with dosing cycles of the anti-tumor agent(s).

In one embodiment, administration frequency of the pharmaceutical composition is normally once or more times per cycle. The frequency is altered each cycle. In another embodiment, the pharmaceutical composition is administered 3 times in the first cycle. In another embodiment, the pharmaceutical composition is administered once a cycle in the second or later cycle. In preferred embodiment, the pharmaceutical composition is administered 3 times in the first cycle, and once a cycle in the second or later cycle.

In one embodiment, administration date in a cycle is arranged by one of ordinary skill in the art. In another embodiment, the date is on days 1, 8 and 15 in the first cycle, and on day 1 in the second or later cycle.

In one embodiment, an amount of obinutuzumab per administration is altered by one of ordinary skill in the art within the range of 1 to 2000 mg. In a specific embodiment, the pharmaceutical composition is intravenously drip infused at 1000 mg of obinutuzumab per administration.

In one embodiment, the pharmaceutical composition is given according to the administration speeds of the following (a) and (b) in two or more cycles.
(a) the maximum administration speed in the first cycle is equal to or more than 200 mg of obinutuzumab an hour, preferably equal to or more than 300 mg of obinutuzumab an hour, or more preferably equal to or more than 400 mg of obinutuzumab an hour.
(b) the maximum administration speed in the second or later cycle is equal to or more than 700 mg of obinutuzumab an hour, preferably equal to or more than 800 mg of obinutuzumab an hour, or more preferably equal to or more than 900 mg of obinutuzumab an hour.

In another embodiment of the above (a), the maximum administration speed in the first cycle is preferably equal to or more than 300 mg of obinutuzumab an hour, or more preferably equal to or more than 400 mg of obinutuzumab an hour.

In another embodiment of the above (b), the maximum administration speed in the second or later cycle is preferably equal to or more than 800 mg of obinutuzumab an hour, or more preferably equal to or more than 900 mg of obinutuzumab an hour. In the embodiment, a duration per administration in the second or later cycle is preferably within 180 minutes. The duration is preferably within 150 minutes, more preferably within 120 minutes, or the most preferably within 90 minutes.

In one embodiment, the first administration in the first cycle is initiated at a speed of 50 mg of obinutuzumab an hour. The second or later administration in the first cycle is initiated at a speed of 100 mg of obinutuzumab an hour.

In one embodiment, the administration speed in the second or later cycle is increased to 700 mg of obinutuzumab an hour or faster, preferably to 800 mg of obinutuzumab an hour or faster, more preferably up to 900 mg of obinutuzumab an hour.

In other embodiment, when the pharmaceutical composition is combined with anti-tumor agent(s), at least one agent is appropriately chosen from heretofore known agents. The other anti-tumor agent is at least one selected from CHOP, CVP, bendamustine, fludarabine, lenalidomide, an anti-PD-1 antibody, and an anti-PD-L1 antibody.

In one embodiment, maintenance monotherapy by obinutuzumab is performed as an additional therapy. The maintenance monotherapy is performed every two months for two years after treatment by the pharmaceutical composition, the treatment is referred to as ‘induction therapy’ in this case.

### III. Production Method

Obinutuzumab can be manufactured by artisan of ordinary skill according to known methods as shown in WO2005/044859. The pharmaceutical composition is also manufactured by mixing obinutuzumab with other ingredients by ordinal skill.

This invention also provides use of obinutuzumab in a manufacture of a pharmaceutical composition for treating CD20-positive B-cell lymphoma. The pharmaceutical composition is administered in the same manner as mentioned in ‘II. Pharmaceutical composition’.

### IV. Treatment Method

This invention also provides a method for treating CD20-positive B-cell lymphoma by a pharmaceutical composition comprising obinutuzumab. In the method, the pharmaceutical composition intravenously drip infused at 1000 mg of obinutuzumab per administration, and given according to the administration speeds of the following (a) and (b) in two or more cycles:
(a) the maximum administration speed in the first cycle is equal to or more than 200 mg of obinutuzumab an hour, preferably equal to or more than 300 mg of obinutuzumab an hour, more preferably equal to or more than 400 mg of obinutuzumab an hour;
(b) the maximum administration speed in the second or later cycle is equal to or more than 700 mg of obinutuzumab an hour, preferably equal to or more than 800 mg of obinutuzumab an hour, more preferably equal to or more than 900 mg of obinutuzumab an hour.

A method to use the pharmaceutical composition is the same as mentioned in ‘II. Pharmaceutical composition’.

It hasn't been cleared whether or not SDI is suitable for administration of obinutuzumab into human before the present invention, because possibility that glycoengineering in obinutuzumab causes any abnormal immunological reaction wasn't able to be denied. Overall, it has been found on the basis of GATS study that the aforementioned pharmaceutical composition is safely and tolerably administered, and can reduce treatment burden on patients and medical professions suffered from a long time administration in regular infusion.

### Examples

### <Overview of GATS Clinical Study>

Title: Safety and tolerability of obinutuzumab (GA101) SDI in Japanese non hodgkin's lymphoma patient

Background: Obinutuzumab (GA101, G) is a novel anti-CD20 monoclonal antibody. G-based immunochemotherapy resulted in a clinically meaningful improvement in progression-free survival (PFS) in patients (pts) with follicular lymphoma (FL) (ASH 2016, #6). Regular infusion (RI) of G takes approx. 3-4 hrs. Shortening the duration of administration may be more convenient for pts.

Methods: The GATS study (JapicCTI-152848) included pts with previously untreated CD20-positive B-cell non-Hodgkin's lymphoma. Treatment consisted of 8 cycles (C) of G, plus CHOP on C1-C6 (with additional G on Days 8 and 15 of C1). SDI was conducted from C2, with infusion of G over 90 min. The primary endpoints were tolerability of SDI, pharmacokinetics (PK), and cytokine release. Tolerability was assessed by incidence of infusion-related reactions (IRRs).

Results: Of 36 pts enrolled, 35 pts were treated, including 19 with diffuse large B-cell lymphoma, 13 with FL, and 3 with other histologies. Overall, 17/35 pts (49%) experienced IRRs. All were Grade 1/2 and occurred most commonly on C1 Day 1 (RI). 2 pts started SDI from C3 or C4 due to deviation or AE. Under SDI, 3 IRRs were observed, but all were Grade 1. Serum G level just after C2 under SDI was similar to that in C8. This shows that PK reached a steady state at C2 and was not affected by shortening of administration. Cytokine elevation was observed during the first G infusion, but immediately decreased at end of the infusion.

Conclusions: SDI of G over 90 min was acceptable in Japanese patients. PK and serum cytokine profiles were comparable to those under RI.

### <Detail Description of GATS Clinical Study>

### Study Design and Treatments

This was a phase II, multicenter, open-label, single-arm study conducted in Japan. Eligible patients were aged ≥20 years with previously untreated and histologically confirmed CD20-positive B-cell NHL (DLBCL, FL or marginal zone lymphoma); Eastern Cooperative Oncology Group performance status of 0-2; life expectancy ≥12 months from date of enrollment; adequate cardiovascular function defined as left ventricular ejection fraction ≥50%; adequate organ function defined as hemoglobin ≥9 g/dL, absolute neutrophil count ≥1.5×10⁹ cells/1, peripheral lymphocytes <5.0×10⁹ cells/1 and platelet count ≥75×10⁹ cells/l; serum bilirubin, serum creatinine and prothrombin time or activated partial thromboplastin time ≤1.5 times the site-specific upper limit and hepatic enzymes ≤2.5 times the site-specific upper limit. Patients were also required not to have undergone major surgery or to have received immune suppression therapy, live vaccine or other study drugs in the 4 weeks preceding enrollment; no monoclonal antibody treatment was permitted within the preceding 12 weeks.

Exclusion criteria included prior therapy for NHL (except for nodal biopsy or local irradiation); primary central nervous system (CNS) lymphoma, secondary CNS involvement or leptomeningeal lymphoma; recent (≤4 weeks) history of significant infection, other malignancy or history of autoimmune disease that could affect the results of the present study; ongoing corticosteroid treatment with the equivalent of prednisolone >30 mg/day for any condition other than lymphoma; any prior use of cytotoxic agents or rituximab or any other anti-CD20 antibody; positive tests for hepatitis B surface (HBs) antigen, HBs antibody, hepatitis B core (HBc) antibody, or hepatitis C virus (HCV) antibody; HIV or human T-cell lymphotropic virus type-I and uncontrolled diabetes mellitus. Patients with HBs antibodies clearly attributable to vaccination and who did not test positive for hepatitis B virus DNA regardless of antibody status were permitted to enroll, as were those who tested positive for HCV antibodies but who had HCV RNA-negative status. The study was approved by local Institutional Review Boards and was conducted in accordance with the Declaration of Helsinki and Good Clinical Practice. All patients gave written and informed consent.

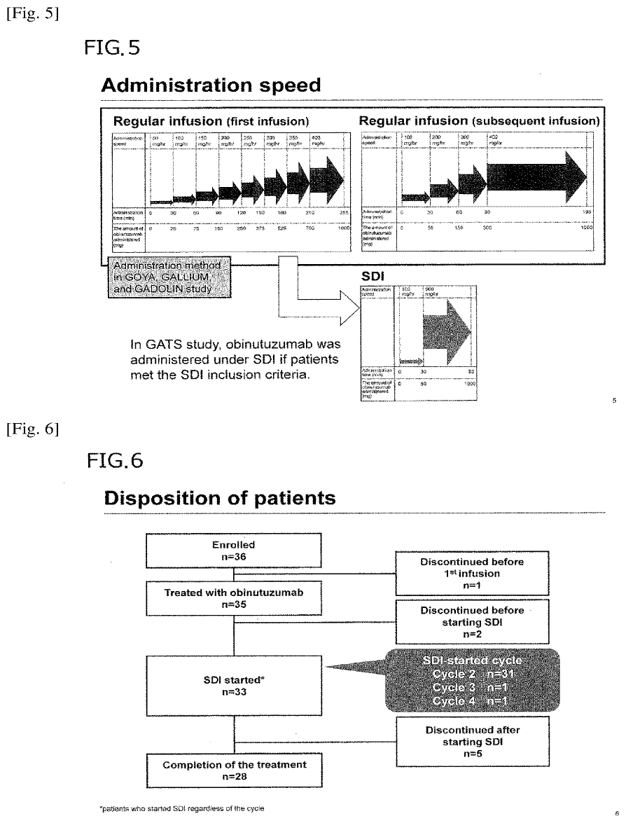

Treatment consisted of eight 21-day cycles of obinutuzumab, given as 1000 mg IV on Days 1, 8 and 15 of Cycle 1 plus standard CHOP on Day 1 of Cycles 1-6 (FIG. 4). Obinutuzumab was administered as RI in Cycle 1 (3-4 h), and then as a 90-min SDI from Cycle 2 in patients who met the SDI criteria (FIG. 5). The SDI criteria were to confirm patient safety at the RI rate and included no grade ≥3 IRR with a causal relationship to obinutuzumab treatment during any of the three RIs in Cycle 1 and a peripheral lymphocyte count <5.0×10⁹ cells/1 before SDI was started. Patients who did not meet these criteria before Cycle 2 could still transition from RI to SDI if they met the criteria in any subsequent cycle.

Standard CHOP consisted of cyclophosphamide 750 mg/m², doxorubicin 50 mg/m² and vincristine 1.4 mg/m² IV on Day 1, and prednisolone 100 mg/day orally or IV on Days 1-5. When obinutuzumab and CHOP were scheduled to be administered on the same day, prednisolone was given prior to the obinutuzumab infusion. Dose reductions to suit the patient's condition were permitted.

### Study Endpoints

The primary endpoints of the study were the incidence of grade ≥3 IRRs in Cycle 2 in patients who started SDI in Cycle 2, serum concentrations and PK parameters of obinutuzumab after SDI up to Day 12 of Cycle 2, and the time courses of cytokine release for tumor necrosis factor alpha (TNFα), interferon gamma (IFNγ) and interleukins 6, 8 and 10 (IL6, IL8 and IL10). IRRs were defined as adverse events (AEs) that were judged by the investigator to be related to obinutuzumab and that were reported during or within 24 h of an infusion.

Secondary endpoints included all other AEs (regardless of relationship to obinutuzumab treatment), IRRs reported during SDI, tumor response at the end of treatment and best response (at any time during follow-up).

The PK analysis population included all patients who received obinutuzumab by SDI on Cycle 2. PK parameters of obinutuzumab for each patient were estimated using non-compartmental analysis (NCA; Phoenix WINNONLIN® version 6.4; CERTARA® USA, Inc.). The following PK parameters were calculated: maximum observed serum concentration (Cmax), area under the serum concentration-time curve from 0 to Day 7 (AUC₀₋₇), elimination half-life (t_{1/2}) and AUC from 0 to last measurable point (AUCₗₐₛₜ).

### Statistical and Analytical Methods

The sample size was based on estimates of the true probability that the incidence of grade ≥3 IRRs in Cycle 2 would exceed 5%. According to the estimates used, on the assumption that there was a grade ≥3 IRR in 1 patient, if 30 patients were recruited, the likelihood that the probability of an IRR would exceed 5% would be 2.2%. This figure was increased to 36 on the assumption that 20% of patients would not be able to make the transition to SDI. The sample size was therefore set at 36 patients.

The incidence rate of grade ≥3 IRRs in Cycle 2 was obtained by dividing the number of patients who developed such reactions by the number of SDI-transition patients. The probability of a grade ≥3 IRR occurring was determined according to the Bayesian approach, using the incidence of grade ≥3 IRRs in Cycle 2 of the GATHER study as the prior distribution. No differences were assumed to exist between Japanese and non-Japanese patients in the probability of developing a grade ≥3 IRR, regardless of infusion rate. We assumed that SDI was adequately tolerated if the true probability of developing a grade ≥3 IRR was ≤5%.

Summary statistics, including arithmetic mean, geometric mean, standard deviation, coefficient of variation, median, minimum and maximum, were calculated for cytokine concentrations at each study visit using serum cytokine concentrations from SDI-transition patients up to Cycle 2. Time courses of cytokine concentrations were also evaluated. The same summary data were generated for PK parameters based on serum obinutuzumab concentrations in SDI transition patients up to Day 12 of Cycle 2. NCA was used. Cycle 2 serum concentrations were compared with additional samples obtained before and after dosing at Cycle 8.

### Patient Population

In total, 36 Japanese patients were enrolled, of whom 35 were treated (safety population; FIG. 6); 28 (80%) completed all eight cycles of treatment. Thirty-one patients started SDI in Cycle 2 (SDI-transition patients), and 2 further patients started SDI in subsequent cycles (1 in Cycle 3 and 1 in Cycle 4) to make a total of 33 SDI-treated patients. Two patients discontinued before starting SDI. Sufficient treatment intensity was achieved; median dose intensity of obinutuzumab was 100%.

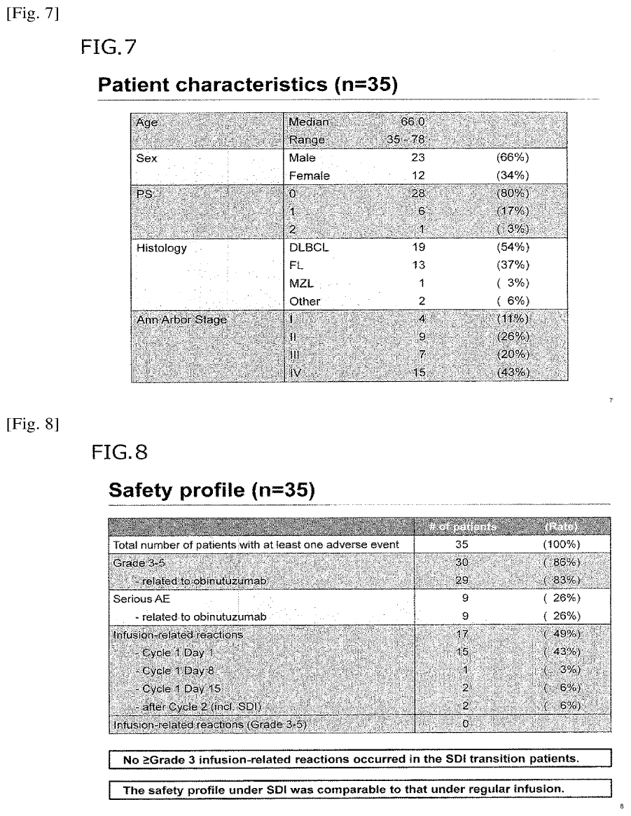

The median age of the patients was 66 years, with just over half of the study population aged between 60 and 70 years (FIG. 7). Approximately two-thirds were male, and the majority of patients had DLBCL (54%) or FL (37%). A fifth of patients (20%) had bone marrow involvement.

### Infusion-Related Reactions

Overall, 17/35 patients (49% of the safety population) experienced a total of 21 IRRs; all were grade 1 or 2, and the majority [ 18/21 IRRs (86%)] occurred during Cycle 1 (in which RI was used). No SDI-associated IRRs occurred in SDI-transition patients in Cycle 2, so it was not possible to estimate the likelihood that the true probability of a grade ≥3 IRR in SDI-transition patients in Cycle 2 would exceed the 5% level inferred using the GATHER study data as the prior distribution. Furthermore, the likelihood that the true probability of a grade ≥3 IRR in SDI transition patients in Cycle 2 would exceed the 5% level inferred using the non-informative prior distribution was 0.05%. There were reports of two patients with IRRs during the SDI in Cycles 6, 7 and 8; all were of grade 1 severity (1 patient experienced nasopharyngitis in Cycle 6, and another experienced headache in Cycles 7 and 8 and palpitations in Cycle 7).

### Other Safety and Tolerability Endpoints

AEs were observed in all 35 patients (FIG. 8). All patients had at least one AE that was judged by the investigator to be treatment-related. Grade ≥3 AEs were observed in 30 patients (86%) and were judged treatment-related in 29 patients (83%). Blood and lymphatic system disorders (neutropenia, leukopenia and thrombocytopenia) were among the treatment-related grade ≥3 AEs most frequently reported. Serious AEs were reported in nine patients (26%). All were judged treatment-related.

There were no AEs leading to death (grade 5) during the study. Obinutuzumab treatment was stopped in three patients because of AEs: one case each of infected dermal cyst, bronchiolitis and aspiration pneumonia. Aspiration pneumonia was not treatment-related. AEs leading to dose reduction or interruption of obinutuzumab treatment occurred in three patients, while AEs leading to dose reduction or interruption of any study drug occurred in nine patients. AEs leading to interruption of any study medication (n=4) were neutropenia, cellulitis, IRR, cerebral infarction or pneumonitis (1 each). Dose reduction of any study medication (n=7) was due to neutropenia/neutrophil count decreased (n=4), leukopenia/white blood cell count decreased (n=3), thrombocytopenia/platelet count decreased (n=3), alanine aminotransferase increased, aspartate aminotransferase increased, neuropathy peripheral, peripheral sensory neuropathy or steroid withdrawal syndrome (1 each).

### Pharmacokinetics

The serum obinutuzumab concentrations of the SDI transition patients in GATS study followed a similar time course to that of patients under regular infusion in the GOYA study (FIG. 10).

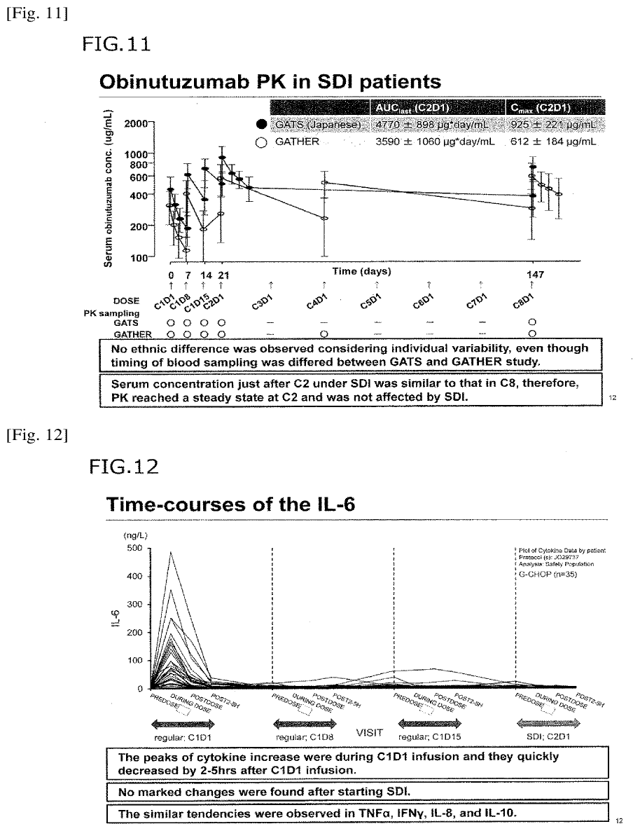

The mean serum obinutuzumab concentration at Cycle 8 was similar to that in Cycle 2 in 17 evaluable SDI-transition patients. This indicates that steady-state PK were attained at Cycle 2 and were not affected by the reduced duration of infusion. The mean±standard deviation AUCₗₐₛₜ (AUC_{7day}) was 4 770±898 μg day/ml at Cycle 2 (vs. 3590±1060 μg day/ml at Cycle 8 in GATHER) (FIG. 11). The mean t_{1/2} was 15.4 ±7.55 days (based on 17 evaluable SDI patients; vs. 23.0±15 days in GATHER). The AUCₗₐₛₜ (AUC_{11day}) value on Day 1 of Cycle 2 was 6790±1450 μg day/ml, with a Cmax of 925±221 μg/ml.

### Cytokines

For all 35 patients (including the 31 SDI-transition patients), cytokine elevations were observed during the first obinutuzumab infusion but were followed by an immediate decrease 2-5 h after the end of the infusion (FIG. 12 shows the case of IL-6). No relevant changes were observed after starting SDI. There was also a rapid depletion in CD19-positive B-cells after the first obinutuzumab infusion. Counts decreased to <0.07×10⁹ cells/1 and remained at this level for the duration of the study.

### Efficacy

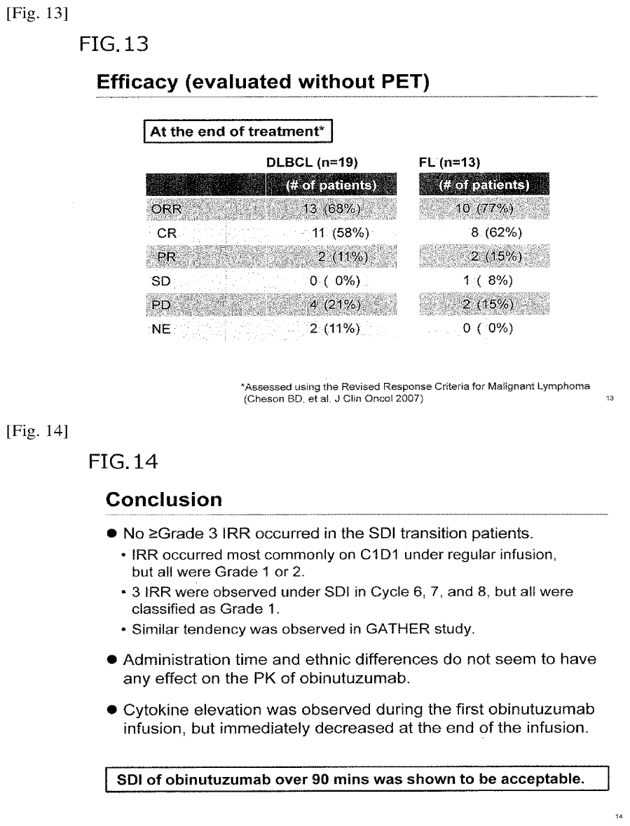

The overall response rate on computed tomography-based assessment at the end of treatment was 77% ( 10/13) in patients with FL and 68% ( 13/19) in patients with DLBCL (including complete and partial responses; FIG. 13). The best overall responses were 92% ( 12/13) and 79% ( 15/19), respectively. Complete responses, CR, (without positron emission tomography scanning) were obtained in 8 of 13 patients with FL (62%) and 11 of 19 patients with DLBCL (58%) at the end of treatment, and in 8 of 13 patients with FL (62%) and 12 of 19 patients with DLBCL (63%) in the best complete response evaluation.

The current study aimed to investigate the tolerability (in particular the rate of IRRs), PK and cytokine release profile of SDI of obinutuzumab plus CHOP chemotherapy in patients with untreated CD20-positive B-cell NHL. The vast majority of IRRs with obinutuzumab plus CHOP were observed in Cycle 1 of treatment, during which RI was used. No IRRs of any grade were observed during Cycle 2, and only two patients experienced IRRs in subsequent cycles during treatment with obinutuzumab by SDI, which were all grade 1.

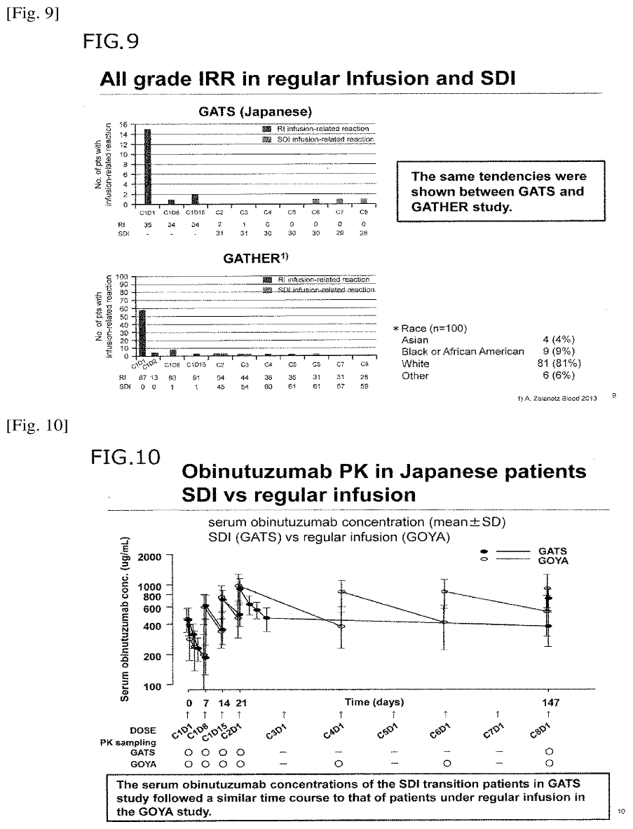

The observed rate of IRRs (49%) is concordant with other reports of obinutuzumab given by RI. Although this is not a direct comparison, it suggests that there is no increased risk of IRRs in patients treated with SDI obinutuzumab. In the phase III GALLIUM trial of obinutuzumab- vs. rituximab-based immunochemotherapy in 1202 previously untreated patients with FL, IRRs were the most common any-grade AEs (68% of obinutuzumab chemotherapy-treated patients) and grade ≥3 AEs (12% of obinutuzumab chemotherapytreated patients) and typically occurred during the first infusion. Similarly, in the phase III GOYA study of 1418 patients with untreated DLBCL, IRRs occurred in 45% (any grade) and 10% (grade ≥3) of patients receiving obinutuzumab with CHOP. In the phase Ib GAUDI study, IRRs occurred in 18 of 28 patients (64%) receiving obinutuzumab plus CHOP; although this occurrence is more common than in the current study, IRRs were also mainly restricted to the first infusion, and grade 3-4 IRRs were infrequent, occurring in two patients (7%). IRRs have also predominated in studies in patients with B-cell malignancies in which obinutuzumab has been trialed as monotherapy, with the majority of reactions being grade 1 or 2. Notably, in the GATHER study in 100 mainly non-Asian patients with DLBCL who received obinutuzumab plus CHOP, no grade ≥3 IRRs were noted in patients who received SDI over 120 or 90 min. The pattern of IRRs seen in GATHER was similar to GATS, with most reactions (77%) occurring during Cycle 1 (during which RI was given) (FIG. 9). Other safety and tolerability findings were similar between the GATHER and GATS populations. No new safety signals were identified in the current study.

PK and serum cytokine data were also found to be comparable with the results of the GATHER study. Exposure to obinutuzumab after SDI was also of the same order in the current study as in GATHER, with similar AUC₀₋₇ and t_{1/2} values. The AUCₗₐₛₜ value reported in the present study from Day 1 of Cycle 2 (4770±885 μg day/ml) is also of the same order of the AUCₗₐₛₜ reported by Ogura et al. on Day 8 of Cycle 1 in patients who received obinutuzumab 800 mg (4190±1190 μg day/ml) or 1200 mg (6540±1070 μg day/ml) in their dose-finding phase I study in 12 Japanese patients with relapsed or refractory B-cell NHL.

Patterns of inflammatory cytokine release, with rapid peaking during the first infusion followed by a rapid reduction and stabilization at baseline levels, were also similar to previous reports. The phase II GAUSS study in 175 patients with relapsed indolent B-cell NHL showed peak cytokine levels of IL6, IL8, IL10, TNFα and IFNγ that were notably elevated during the first infusion of obinutuzumab but then returned to baseline without any increase during subsequent infusions. The same pattern was reported in the phase I/II GAUGUIN study in the cohort of 33 patients with relapsed or refractory CLL. We note, as did the authors of GAUGUIN, that these early elevations in inflammatory cytokine levels coincide with the increased rates of reporting of IRRs during the first cycle of treatment in GATS and the other studies mentioned. The pattern of CD19-positive B-cell response was also similar to previous reports. Ogura et al. showed a rapid reduction after the first infusion of obinutuzumab, with the nadir achieved in most patients after infusion on Day 1. The same rapid Bcell depletion was reported in Cycle 1 of GAUGUIN in the CLL cohort and the indolent B-cell NHL cohort.

Overall and complete response rates at the end of treatment in patients with DLBCL (68 and 58%, respectively) were of the same order as those obtained in GATHER (82 and 55%, respectively), in which SDI was also used for patients with DLBCL. There are no data available yet for response rates in FL patients treated with obinutuzumab using SDI for comparison.

A limitation of the current study lies in the small patient population; a much larger sample would be required for definitive assessment of safety of obinutuzumab SDI, although the results obtained do appear concordant with previous findings in both Japanese patients and those undergoing treatment with SDI. We note also that the GATS study lacked a control arm in which, for example, patients might have received a conventional full set of cycles of obinutuzumab by RI in addition to CHOP.

In conclusion, obinutuzumab given by SDI was well tolerated in this Japanese patient cohort (FIG. 14). No SDI-associated IRRs were observed in the second cycle of treatment (i.e. the first SDI cycle); a small number of IRRs were observed with SDI in later cycles but were tolerable and manageable. The rate of IRRs was in line with findings from other studies of obinutuzumab given by RI, indicating that there is no increased risk of IRRs when obinutuzumab is given by SDI. Overall, the findings suggest that obinutuzumab can be administered safely by SDI.

1. A pharmaceutical composition for treating CD20-positive B-cell lymphoma comprising obinutuzumab, which is intravenously drip infused at 1000 mg of obinutuzumab per administration, and given according to the administration speeds of the following (a) and (b) in two or more cycles:
(a) the maximum administration speed in the first cycle is equal to or more than 200 mg of obinutuzumab an hour, preferably equal to or more than 300 mg of obinutuzumab an hour, more preferably equal to or more than 400 mg of obinutuzumab an hour;
(b) the maximum administration speed in the second or later cycle is equal to or more than 700 mg of obinutuzumab an hour, preferably equal to or more than 800 mg of obinutuzumab an hour, more preferably equal to or more than 900 mg of obinutuzumab an hour.

2. The pharmaceutical composition according to claim 1, wherein a duration per administration in the second or later cycle is within 180 minutes, preferably within 150 minutes, more preferably within 120 minutes, the most preferably within 90 minutes.

3. The pharmaceutical composition according to claim 1 or 2, which is administered 3 times in the first cycle, and once a cycle in the second or later cycle.

4. The pharmaceutical composition according to claim 3, wherein the first administration in the first cycle is initiated at a speed of 50 mg of obinutuzumab an hour, and the second or later administration in the first cycle is initiated at a speed of 100 mg of obinutuzumab an hour.

5. The pharmaceutical composition according to any one of claims 1 to 4, wherein the administration speed in the second or later cycle is increased to 700 mg of obinutuzumab an hour or faster, preferably to 800 mg of obinutuzumab an hour or faster, more preferably up to 900 mg of obinutuzumab an hour.

6. The pharmaceutical composition according to any one of claims 1 to 5, wherein, in (b), the pharmaceutical composition is given according to at least one of the following (c) to (e) conditions:
(c) if no infusion reaction of Grade 3 or above appeared with the last three administrations, and the number of lymphocytes in peripheral blood before administration is less than 5000/μL, administration is carried out at 100 mg/hour for 30 minutes. If no infusion reaction is observed all that time, the speed can be increased to 900 mg/hour. Depending on the condition of the patient, the speed is decreased to, for example, the administration speed in cycle 1, as appropriate.
(d) if an infusion reaction of Grade 1/2 appeared, administration is restarted at half the speed before administration was stopped. If no infusion reaction is observed in 30 minutes, the speed can be increased to 900 mg/hour.
(e) if an infusion reaction of Grade 3, administration is restarted at 200 mg/hour or lower. If no infusion reaction is observed in 30 minutes, the speed can be increased by 50 mg/hour every 30 minutes to a maximum of 400 mg/hour.

7. The pharmaceutical composition according to any one of claims 1 to 6, which is administered on days 1, 8 and 15 in the first cycle, and on day 1 in the second or later cycle.

8. The pharmaceutical composition according to any one of claims 1 to 7, wherein each cycle is 3 weeks.

9. The pharmaceutical composition according to any one of claims 1 to 7, which is used in combination with at least one of other anti-tumor agents, and whose administration cycle is synchronized with a dosing cycle of said at least one of other anti-tumor agents, wherein the dosing cycle is 4 weeks a cycle.

10. The pharmaceutical composition according to claim 9, wherein said at least one of other anti-tumor agents is selected from CHOP, CVP, bendamustine, fludarabine, lenalidomide, an anti-PD-1 antibody, and an anti-PD-L1 antibody.

11. The pharmaceutical composition according to any one of claims 1 to 10, wherein the pharmaceutical composition is given every two months for two years as maintenance monotherapy after said two or more cycles.

12. The pharmaceutical composition according to any one of claims 1 to 11, wherein the obinutuzumab concentration in infusion fluid when intravenously drip infused is 10 to 40 mg/mL, preferably 20 to 30 mg/mL, more preferably 25 mg/mL.

13. The pharmaceutical composition according to any one of claims 1 to 12, further comprising a trehalose hydrate, L-histidine, L-histidine hydrochloride hydrate, or polyoxyethylene (160) polyoxypropylene (30) glycol as an additive.

14. Use of obinutuzumab in a manufacture of a pharmaceutical composition for treating CD20-positive B-cell lymphoma comprising obinutuzumab, wherein the composition is intravenously drip infused at 1000 mg of obinutuzumab per administration, and given according to the administration speeds of the following (a) and (b) in two or more cycles:
(a) the maximum administration speed in the first cycle is equal to or more than 200 mg of obinutuzumab an hour, preferably equal to or more than 300 mg of obinutuzumab an hour, more preferably equal to or more than 400 mg of obinutuzumab an hour;
(b) the maximum administration speed in the second or later cycle is equal to or more than 700 mg of obinutuzumab an hour, preferably equal to or more than 800 mg of obinutuzumab an hour, more preferably equal to or more than 900 mg of obinutuzumab an hour.

15. A method for treating CD20-positive B-cell lymphoma by a pharmaceutical composition comprising obinutuzumab, wherein the composition is intravenously drip infused at 1000 mg of obinutuzumab per administration, and given according to the administration speeds of the following (a) and (b) in two or more cycles:
(a) the maximum administration speed in the first cycle is equal to or more than 200 mg of obinutuzumab an hour, preferably equal to or more than 300 mg of obinutuzumab an hour, more preferably equal to or more than 400 mg of obinutuzumab an hour;
(b) the maximum administration speed in the second or later cycle is equal to or more than 700 mg of obinutuzumab an hour, preferably equal to or more than 800 mg of obinutuzumab an hour, more preferably equal to or more than 900 mg of obinutuzumab an hour.
